(19) United States
(12) Patent Application Publication
(10) Pub. No.: US 2024/0132960 A1
(43) Pub. Date: Apr. 25, 2024

(54) METHODS FOR DETECTION OF DONOR-DERIVED CELL-FREE DNA IN TRANSPLANT RECIPIENTS OF MULTIPLE ORGANS

(71) Applicant: Natera, Inc., San Carlos, CA (US)

(72) Inventors: Zachary DEMKO, San Francisco, CA (US); Ebad AHMED, San Jose, CA (US); Ryan SWENERTON, Millbrae, CA (US); Paul VAN HUMMELEN, San Francisco, CA (US)

(73) Assignee: Natera, Inc., San Carlos, CA (US)

(21) Appl. No.: 18/279,006

(22) PCT Filed: Feb. 24, 2022
(86) PCT No.: PCT/US2022/017707

§ 371 Date: Aug. 24, 2023

Related U.S. Application Data
(60) Provisional application No. 63/153,800, filed on Feb. 25, 2021.

(51) Int. Cl. C12Q 1/6883; C12Q 1/6869

(52) U.S. Cl. CPC C12Q 1/6883; C12Q 1/6869; C12Q 2600/156

(57) ABSTRACT

The present disclosure provides methods of amplifying and sequencing DNA, comprising: extracting cell-free DNA from a blood, plasma, serum or urine sample of a transplant recipient who has received transplantation of one or more organs including simultaneous or sequential transplantation of multiple organs, wherein the extracted cell-free DNA comprises donor-derived cell-free DNA and recipient-derived cell-free DNA; performing targeted amplification at 200-50,000 target loci in a single reaction volume using 200-50,000 primer pairs, wherein the target loci comprise polymorphic loci and non-polymorphic loci; sequencing the amplification products by high-throughput sequencing to obtain a sequencing reads and quantifying the amount of donor-derived cell-free DNA and the amount of total cell-free DNA based on the sequencing reads; and determining whether the amount of donor-derived cell-free DNA or a function thereof exceeds a cutoff threshold indicating transplant rejection or graft injury.

### BACKGROUND

Rapid detection of graft injury and/or rejection remains a challenge for transplant recipients of multiple organs, either from simultaneous multi-organ transplantation or sequential transplantations. Conventional biopsy-based tests are invasive and costly and possibly lead to late diagnosis of transplant injury and/or rejection. Therefore, there is a need for a non-invasive transplantation rejection test for transplant recipients of multiple organs that is more sensitive and more specific than conventional biopsy-based tests.

### SUMMARY

The present invention relates to methods of amplifying and sequencing DNA, comprising: extracting cell-free DNA from a blood, plasma, serum or urine sample of a transplant recipient who has received transplantation of one or more organs including simultaneous or sequential transplantation of multiple organs, wherein the extracted cell-free DNA comprises donor-derived cell-free DNA and recipient-derived cell-free DNA; performing targeted amplification at 200-50,000 target loci in a single reaction volume using 200-50,000 primer pairs, wherein the target loci comprise polymorphic loci and non-polymorphic loci; sequencing the amplification products by high-throughput sequencing to obtain a sequencing reads and quantifying the amount of donor-derived cell-free DNA and the amount of total cell-free DNA based on the sequencing reads; and determining whether the amount of donor-derived cell-free DNA or a function thereof exceeds a cutoff threshold indicating transplant rejection or graft injury.

### DETAILED DESCRIPTION

Sigdel et al., “Optimizing Detection of Kidney Transplant Injury by Assessment of Donor-Derived Cell-Free DNA via Massively Multiplex PCR,” J. Clin. Med. 8(1):19 (2019), is incorporated herein by reference in its entirety.

WO2020/010255, titled “Methods for Detection of Donor-Derived Cell-Free DNA” and filed on Jul. 3, 2019 as PCT/US2019/040603, is incorporated herein by reference in its entirety.

U.S. Prov. Appl. No. 63/031,879, titled “Improved Methods for Detection of Donor Derived Cell-Free DNA” and filed on May 29, 2020, is incorporated herein by reference in its entirety.

The present invention relates to methods of amplifying and sequencing cell-free DNA extracted from a biological sample of a transplant recipient who has received transplantation of one or more organs including simultaneous or sequential transplantation of multiple organs, which is useful for determine transplant rejection or graft injury. In some embodiments, the method comprises (a) extracting cell-free DNA from a blood, plasma, serum or urine sample of the transplant recipient, wherein the extracted cell-free DNA comprises donor-derived cell-free DNA and recipient-derived cell-free DNA; (b) performing targeted amplification at 200-50,000 target loci in a single reaction volume using 200-50,000 primer pairs, wherein the target loci comprise polymorphic loci and non-polymorphic loci; (c) sequencing the amplification products by high-throughput sequencing to obtain a sequencing reads and quantifying the amount of donor-derived cell-free DNA and the amount of total cell-free DNA based on the sequencing reads; and (d) determining whether the amount of donor-derived cell-free DNA or a function thereof exceeds a cutoff threshold indicating transplant rejection or graft injury.

In some embodiments, the transplant donor is a human subject. In some embodiments, the transplant donor is a non-human mammalian subject (e.g., pig). In some embodiments, the transplant recipient is a human subject.

In some embodiments, the transplant recipient has received a one or more transplanted organs, including but limited to pancreas, kidney, liver, heart, intestinal, thymus, hematopoietic cells, and uterus.

In some embodiments, a plurality of organs are from the same transplant donor. In some embodiments, a plurality of organs are from different transplant donors.

In some embodiments, the transplant recipient has received simultaneous transplantation of a plurality of organs. In some embodiments, the transplant recipient has received sequential transplantation of a plurality of organs.

In some embodiments, the transplant recipient has received simultaneous transplantation of kidney and pancreas (SPK). In some embodiments, the transplant recipient has received sequential transplantation of kidney and pancreas (PAK).

In some embodiments, the transplant recipient has received simultaneous transplantation of kidney and liver. In some embodiments, the transplant recipient has received simultaneous transplantation of kidney and heart. In some embodiments, the transplant recipient has received simultaneous transplantation of kidney and lung. In some embodiments, the transplant recipient has received simultaneous transplantation of a pancreas and liver. In some embodiments, the transplant recipient has received simultaneous transplantation of heart and lung.

In some embodiments, the transplant recipient has received sequential transplantation of kidney and liver. In some embodiments, the transplant recipient has received sequential transplantation of kidney and heart. In some embodiments, the transplant recipient has received sequential transplantation of kidney and lung. In some embodiments, the transplant recipient has received sequential transplantation of a pancreas and liver. In some embodiments, the transplant recipient has received sequential transplantation of heart and lung.

In some embodiments, the cutoff threshold is a percentage of donor-derived cell-free DNA out of the amount of total cell-free DNA, such as 0.1%, 0.15%, 0.2%, 0.25%, 0.3%, 0.35%, 0.4%, 0.45%, or 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%. In some embodiments, the cutoff threshold is adjusted according to the type of organs transplanted. In some embodiments, the cutoff threshold is adjusted according to the number of organs transplanted.

In some embodiments, the cutoff threshold for a transplant recipient who has received a kidney transplant is 1.0%, 1.1%, 1.2%, 1.3%, 1.4%, 1.5%, 1.6%, 1.7%, 1.8%, 1.9%, or 2.0%, of the amount of donor-derived cell-free DNA out of the amount of total cell-free DNA.

In some embodiments, the cutoff threshold for a transplant recipient who has received a heart transplant is 0.1%, 0.15%, 0.2%, 0.25%, 0.3%, 0.35%, 0.4%, 0.45%, or 0.5%, of the amount of donor-derived cell-free DNA out of the amount of total cell-free DNA.

In some embodiments, the cutoff threshold is an amount of donor-derived cell-free DNA or a function thereof. In some embodiments, the cutoff threshold is expressed as a relative quantity or absolute quantity of dd-cfDNA. In some embodiments, the cutoff threshold is expressed as a relative quantity or absolute quantity of dd-cfDNA per volume unit of the blood sample. In some embodiments, the cutoff threshold is expressed as a relative quantity or absolute quantity of dd-cfDNA per volume unit of the blood sample, multiplied or divided, by body mass, BMI, or blood volume of the transplant recipient.

In some embodiment, a two-threshold algorithm which combines both dd-cfDNA (%) and absolute quantity of dd-cfDNA (copies/mL) is applied with the goal of improving test sensitivity, particularly through improved detection in cases where cfDNA levels are high. In some embodiment, in the new two-threshold algorithm, the dd-cfDNA fraction cut-off is 0.1%, 0.15%, 0.2%, 0.25%, 0.3%, 0.35%, 0.4%, 0.45%, or 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%, and the dd-cfDNA quantity cut-off is 10 cp/mL, or 15 cp/m, or 20 cp/mL, or 25 cp/mL, or 30 cp/mL, or 35 cp/mL, or 40 cp/mL, or 45 cp/mL, or 50 cp/mL, or 60 cp/mL, or 70 cp/mL, or 80 cp/mL, or 90 cp/mL, or 100 cp/mL, or 110 cp/mL, or 120 cp/mL, or 130 cp/mL, or 140 cp/mL, or 150 cp/mL. In some embodiments, samples exceeding either threshold were considered at high risk for AR or graft injury. In some embodiments, samples exceeding both thresholds were considered at high risk for AR or graft injury.

In some embodiments, the targeted amplification comprises PCR, and the primer pairs comprise 200-50,000, 500-20,000, or 1,000-10,000, or 200-500, or 500-1,000, or 1,000-2,000, or 2,000-5,000, or 5,000-10,000, or 10,000-20,000, or 20,000-50,000 pairs of forward and reverse PCR primers. In some embodiments, the targeted amplification comprises performing targeted amplification at 500-20,000, or 1,000-10,000, or 200-500, or 500-1,000, or 1,000-2,000, or 2,000-5,000, or 5,000-10,000, or 10,000-20,000, or 20,000-50,000 target loci in a single reaction volume using 500-20,000, or 1,000-10,000, or 200-500, or 500-1,000, or 1,000-2,000, or 2,000-5,000, or 5,000-10,000, or 10,000-20,000, or 20,000-50,000 primer pairs to obtain amplification products.

In some embodiments, the target loci comprise single nucleotide polymorphisms (SNPs).

In some embodiments, the method further comprises attaching tags to the amplification products prior to performing high-throughput sequencing, wherein the tags comprise sequencing-compatible adaptors.

In some embodiments, the method further comprises attaching tags to the extracted cell-free DNA prior to performing targeted amplification, wherein the tags comprise adaptors for amplification.

In some embodiments, the tags comprise sample-specific barcodes, and wherein the method further comprises pooling the amplification products from a plurality of samples prior to high-throughput sequencing and sequencing the pool of amplification products together in a single run during the high-throughput sequencing.

In some embodiments, the method further comprises repeating steps (a)-(d) longitudinally for the same transplant recipient, and determining a longitudinal change in the amount of donor-derived cell-free DNA or a function thereof in said transplant recipient.

In some embodiments, the method further comprises adjusting immunosuppressive therapy based on the longitudinal change in the amount of donor-derived cell-free DNA or a function thereof in the transplant recipient. In some embodiments, the method further comprises increasing immunosuppressive therapy in view of increased amount of donor-derived cell-free DNA or a function thereof in the transplant recipient. In some embodiments, the method further comprises decreasing immunosuppressive therapy in view of decreased amount of donor-derived cell-free DNA or a function thereof in the transplant recipient.

In some embodiments, the method is performed without prior knowledge of donor genotypes. In some embodiments, the method does not comprise genotyping transplant donor(s).

The methods described herein assess all types of transplant rejection or graft injury with great precision. From a single blood draw, which may include two or more tubes of blood, certain embodiments of the methods described herein measure the amount of donor cfDNA from the multiple transplanted organ in the patient's blood. Using a large number of single-nucleotide polymorphisms (SNP) (e.g., more than 13,000 SNPs) and advanced bioinformatics, these embodiments can differentiate donor and recipient cfDNA to provide a result such as a percentage of dd-cfDNA in a transplant recipient's blood or an amount of donor-derived cell-free DNA or a function thereof.

In some embodiments, for example, tracer DNA, or internal calibration DNA, refers to a composition of DNA for which one or more of the following is known advance—length, sequence, nucleotide composition, quantity, or biological origin. The tracer DNA can be added to a biological sample derived from a human subject to help estimate the amount of total cfDNA in said sample. It can also be added to reaction mixtures other than the biological sample itself.

In some embodiments, for example, single nucleotide polymorphism (SNP) refers to a single nucleotide that may differ between the genomes of two members of the same species. The usage of the term does not imply any limit on the frequency with which each variant occurs.

In some embodiments, for example, sequence refers to a DNA or RNA sequence or a genetic sequence. It may refer to the primary, physical structure of the DNA or RNA molecule or strand in an individual. It may refer to the sequence of nucleotides found in that DNA or RNA molecule, or the complementary strand to the DNA or RNA molecule. It may refer to the information contained in the DNA or RNA molecule as its representation in silico.

In some embodiments, for example, locus refers to a particular region of interest on the DNA or RNA of an individual and includes without limitation one or more SNPs, the site of a possible insertion or deletion, or the site of some other relevant genetic variation. Disease-linked SNPs may also refer to disease-linked loci.

In some embodiments, for example, polymorphic allele, also “polymorphic locus,” refers to an allele or locus where the genotype varies between individuals within a given species. Some examples of polymorphic alleles include single nucleotide polymorphisms (SNPs), short tandem repeats, deletions, duplications, and inversions.

In some embodiments, for example, allele refers to the nucleotides or nucleotide sequence occupying a particular locus.

In some embodiments, for example, genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

In some embodiments, for example, genetic material also “genetic sample” refers to physical matter, such as tissue or blood, from one or more individuals comprising nucleic acids (e.g., comprising DNA or RNA)

In some embodiments, for example, allelic data refers to a set of genotypic data concerning a set of one or more alleles. It may refer to the phased, haplotypic data. It may refer to SNP identities, and it may refer to the sequence data of the nucleic acid, including insertions, deletions, repeats and mutations.

In some embodiments, for example, allelic state refers to the actual state of the genes in a set of one or more alleles. It may refer to the actual state of the genes described by the allelic data.

In some embodiments, for example, allelic ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.

In some embodiments, for example, allele count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

In some embodiments, for example, primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow amplification such as PCR amplification. A primer may also contain a molecular barcode. A primer may contain a random region that differs for each individual molecule.

In some embodiments, for example, hybrid capture probe refers to any nucleic acid sequence, possibly modified, that is generated by various methods such as PCR or direct synthesis and intended to be complementary to one strand of a specific target DNA or RNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.

In some embodiments, for example, sequence read refers to data representing a sequence of nucleotide bases that were measured using a clonal sequencing method. Clonal sequencing may produce sequence data representing single, or clones, or clusters of one original DNA or RNA molecule. A sequence read may also have associated quality score at each base position of the sequence indicating the probability that nucleotide has been called correctly.

In some embodiments, for example, mapping a sequence read is the process of determining a sequence read's location of origin in the genome sequence of a particular organism. The location of origin of sequence reads is based on similarity of nucleotide sequence of the read and the genome sequence.

In some embodiments, for example, DNA or RNA of donor origin refers to DNA or RNA that was originally part of a cell whose genotype was essentially equivalent to that of the transplant donor. The donor can be a human or a non-human mammalian (e.g., pig).

In some embodiments, for example, DNA or RNA of recipient origin refers to DNA or RNA that was originally part of a cell whose genotype was essentially equivalent to that of the transplant recipient.

In some embodiments, for example, transplant recipient plasma refers to the plasma portion of the blood from a female from a patient who has received an allograft or xenograft, e.g., an organ transplant recipient.

In some embodiments, for example, preferential enrichment of DNA or RNA that corresponds to a locus, or preferential enrichment of DNA or RNA at a locus, refers to any technique that results in the percentage of molecules of DNA or RNA in a post-enrichment DNA or RNA mixture that correspond to the locus being higher than the percentage of molecules of DNA or RNA in the pre-enrichment DNA or RNA mixture that correspond to the locus. The technique may involve selective amplification of DNA or RNA molecules that correspond to a locus. The technique may involve removing DNA or RNA molecules that do not correspond to the locus. The technique may involve a combination of methods. The degree of enrichment is defined as the percentage of molecules of DNA or RNA in the post-enrichment mixture that correspond to the locus divided by the percentage of molecules of DNA or RNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

In some embodiments, for example, amplification refers to a technique that increases the number of copies of a molecule of DNA or RNA.

In some embodiments, for example, selective amplification may refer to a technique that increases the number of copies of a particular molecule of DNA or RNA, or molecules of DNA or RNA that correspond to a particular region of DNA or RNA. It may also refer to a technique that increases the number of copies of a particular targeted molecule of DNA or RNA, or targeted region of DNA or RNA more than it increases non-targeted molecules or regions of DNA or RNA. Selective amplification may be a method of preferential enrichment.

In some embodiments, for example, universal priming sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.

In some embodiments, for example, universal adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

In some embodiments, for example, targeting refers to a method used to selectively amplify or otherwise preferentially enrich those molecules of DNA or RNA that correspond to a set of loci in a mixture of DNA or RNA.

### Analysis of Donor-Derived Cell-Free DNA for Monitoring Transplant Rejection or Graft Injury

In one aspect, the present invention relates to a method of quantifying the amount of donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 500-50,000 target loci in a single reaction volume using 500-50,000 primer pairs, wherein the target loci comprise polymorphic loci and non-polymorphic loci, and wherein each primer pair is designed to amplify a target sequence of no more than 100 bp; and quantifying the amount of donor-derived cell-free DNA in the amplification products.

In another aspect, the present invention relates to a method of quantifying the amount of donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA, and wherein the extracting step comprises size selection to enrich for donor-derived cell-free DNA and reduce the amount of recipient-derived cell-free DNA disposed from bursting white-blood cells; performing targeted amplification at 500-50,000 target loci in a single reaction volume using 500-50,000 primer pairs, wherein the target loci comprise polymorphic loci and non-polymorphic loci; and quantifying the amount of donor-derived cell-free DNA in the amplification products.

In another aspect, the present invention relates to a method of detecting donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA.

In some embodiments, the method further comprises performing universal amplification of the extracted DNA. In some embodiments, the universal amplification preferentially amplifies donor-derived cell-free DNA over recipient-derived cell-free DNA that are disposed from bursting white-blood cells.

In some embodiments, the transplant donor is a human subject. In some embodiments, the transplant donor is a non-human mammalian subject (e.g., pig). In some embodiments, the transplant recipient is a mammal. In some embodiments, the transplant recipient is a human.

In some embodiments, the transplant recipient has received a transplant selected from organ transplant, tissue transplant, cell transplant, and fluid transplant. In some embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, hematopoietic cells, and blood transfusion. In some embodiments, the transplant recipient has received SPK transplant.

In some embodiments, the quantifying step comprises determining the percentage of donor-derived cell-free DNA out of the total of donor-derived cell-free DNA and recipient-derived cell-free DNA in the blood sample. In some embodiments, the quantifying step comprises determining the amount of donor-derived cell-free DNA per volume unit of the blood sample.

In some embodiments, the method further comprises detecting the occurrence or likely occurrence of active rejection of transplantation using the quantified amount of donor-derived cell-free DNA. In some embodiments, the method is performed without prior knowledge of donor genotypes.

In some embodiments, each primer pair is designed to amplify a target sequence of about 50-100 bp. In some embodiments, each primer pair is designed to amplify a target sequence of no more than 75 bp. In some embodiments, each primer pair is designed to amplify a target sequence of about 60-75 bp. In some embodiments, each primer pair is designed to amplify a target sequence of about 65 bp.

In some embodiments, the targeted amplification comprises amplifying at least 1,000 polymorphic loci in a single reaction volume. In some embodiments, the targeted amplification comprises amplifying at least 2,000 polymorphic loci in a single reaction volume. In some embodiments, the targeted amplification comprises amplifying at least 5,000 polymorphic loci in a single reaction volume. In some embodiments, the targeted amplification comprises amplifying at least 10,000 polymorphic loci in a single reaction volume.

In some embodiments, method further comprises measuring an amount of one or more alleles at the target loci that are polymorphic loci. In some embodiments, the polymorphic loci and the non-polymorphic loci are amplified in a single reaction.

In some embodiments, the quantifying step comprises detecting the amplified target loci using a microarray. In some embodiments, the quantifying step does not comprise using a microarray.

In some embodiments, the targeted amplification comprises simultaneously amplifying 500-50,000 target loci in a single reaction volume using (i) at least 500-50,000 different primer pairs, or (ii) at least 500-50,000 target-specific primers and a universal or tag-specific primer 500-50,000 primer pairs.

In a further aspect, the present invention relates to a method of determining the likelihood of transplant rejection or graft injury within a transplant recipient, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a greater amount of dd-cfDNA indicates a greater likelihood of transplant rejection or graft injury.

In a further aspect, the present invention relates to a method of diagnosing a transplant within a transplant recipient as undergoing acute rejection, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some embodiments, the transplant rejection is antibody mediated transplant rejection. In some embodiments, the transplant rejection is T cell mediated transplant rejection.

In some embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In a further aspect, the present invention relates to a method of monitoring immunosuppressive therapy in a subject, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a change in levels of dd-cfDNA over a time interval is indicative of transplant status.

In some embodiments, the method further comprising adjusting immunosuppressive therapy based on the levels of dd-cfDNA over the time interval.

In some embodiments, an increase in the levels of dd-cfDNA is indicative of transplant rejection and a need for adjusting immunosuppressive therapy. In some embodiments, no change or a decrease in the levels of dd-cfDNA indicates transplant tolerance or stability, and a need for adjusting immunosuppressive therapy.

In some embodiments, an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection. In some embodiments, the transplant rejection is antibody mediated transplant rejection. In some embodiments, the transplant rejection is T cell mediated transplant rejection.

In some embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In some embodiments, the method does not comprise genotyping the transplant donor and/or the transplant recipient.

In some embodiments, the method further comprises measuring an amount of one or more alleles at the target loci that are polymorphic loci.

In some embodiments, the target loci comprise at least 1,000 polymorphic loci, or at least 2,000 polymorphic loci, or at least 5,000 polymorphic loci, or at least 10,000 polymorphic loci.

In some embodiments, the target loci that are amplified in amplicons of about 50-100 bp in length, or about 50-90 bp in length, or about 60-80 bp in length, or about 60-75 bp in length, or about 65 bp in length.

In some embodiments, the transplant recipient is a human. In some embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some embodiments, the transplant recipient has received SPK transplant.

In some embodiments, the extracting step comprises size selection to enrich for donor-derived cell-free DNA and reduce the amount of recipient-derived cell-free DNA disposed from bursting white-blood cells.

In some embodiments, the universal amplification step preferentially amplifies donor-derived cell-free DNA over recipient-derived cell-free DNA that are disposed from bursting white-blood cells.

In some embodiments, the method comprises longitudinally collecting a plurality of blood samples from the transplant recipient after transplantation, and repeating steps (a) to (e) for each blood sample collected. In some embodiments, the method comprises collecting and analyzing blood samples from the transplant recipient for a time period of about three months, or about six months, or about twelve months, or about eighteen months, or about twenty-four months, etc. In some embodiments, the method comprises collecting blood samples from the transplant recipient at an interval of about one week, or about two weeks, or about three weeks, or about one month, or about two months, or about three months, etc.

In some embodiments, the method has a sensitivity of at least 80%, or at least 85%, or at least 90%, or at least 95%, or at least 98% in identifying acute rejection (AR) over non-AR with a cutoff threshold of 1% dd-cfDNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-cfDNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-cfDNA and a confidence interval of 95%.

In some embodiments, the method has a sensitivity of at least 80%, or at least 85%, or at least 90%, or at least 95%, or at least 98% in identifying AR over normal, stable allografts (STA) with a cutoff threshold of 1% dd-cfDNA and a confidence interval of 95%.

In some embodiments, the method has a specificity of at least 80%, or at least 85%, or at least 90%, or at least 95%, or at least 98% in identifying AR over STA with a cutoff threshold of 1% dd-cfDNA and a confidence interval of 95%.

In some embodiments, the method has an AUC of at least 0.8, or 0.85, or at least 0.9, or at least 0.95, or at least 0.98, or at least 0.99 in identifying AR over STA with a cutoff threshold of 1% dd-cfDNA and a confidence interval of 95%.

In some embodiments, the method has a sensitivity as determined by a limit of blank (LoB) of 0.5% or less, and a limit of detection (LoD) of 0.5% or less. In some embodiments, LoB is 0.23% or less and LoD is 0.29% or less. In some embodiments, the sensitivity is further determined by a limit of quantitation (LoQ). In some embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some embodiments, LoB is equal to or less than 0.04%, LoD is equal to or less than 0.05%, and/or LoQ is equal to the LoD.

In some embodiments, the method has an accuracy as determined by evaluating a linearity value obtained from linear regression analysis of measured donor fractions as a function of the corresponding attempted spike levels, wherein the linearity value is a R2 value, wherein the R2 value is from about 0.98 to about 1.0. In some embodiments, the R2 value is 0.999. In some embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about −0.0001 to about 0.01. In some embodiments, the slope value is approximately 1, and the intercept value is approximately 0.

In some embodiments, the method has a precision as determined by calculating a coefficient of variation (CV), wherein the CV is less than about 10.0%. CV is less than about 6%. In some embodiments, the CV is less than about 4%. In some embodiments, the CV is less than about 2%. In some embodiments, the CV is less than about 1%.

In some embodiments, the AR is antibody-mediated rejection (ABMR). In some embodiments, the AR is T-cell-mediated rejection (TCMR). In some embodiments, the AR is acute cellular rejection (ACR).

Further disclosed herein are methods for detection of transplant donor-derived cell-free DNA (dd-cfDNA) in a sample from a transplant recipient. In some embodiments, in the methods disclosed herein, the transplant recipient is a mammal. In some embodiments, the transplant recipient is a human. In some embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some embodiments, the transplant recipient has received SPK transplant. In some embodiments, the method may be performed on transplant recipients the day of or after transplant surgery, up to a year following transplant surgery.

In some embodiments, disclosed herein is a method of amplifying target loci of donor-derived cell-free DNA (dd-cfDNA) from a blood sample of a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; and c) amplifying the target loci.

In some embodiments, disclosed herein is a method of detecting donor-derived cell-free DNA (dd-cfDNA) in a blood sample from a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting dd-cfDNA in the blood sample. In some embodiments, the probes are labelled with a detectable marker.

In some embodiments, disclosed herein is a method of determining the likelihood of transplant rejection within a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a greater amount of dd-cfDNA indicates a greater likelihood of transplant rejection.

In some embodiments, disclosed herein is a method of diagnosing a transplant within a transplant recipient as undergoing acute rejection, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some embodiments, in the methods disclosed herein, the transplant rejection is antibody mediated transplant rejection. In some embodiments, the transplant rejection is T cell mediated transplant rejection. In some embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In some embodiments, disclosed herein is a method of monitoring immunosuppressive therapy in a subject, the method comprising a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a change in levels of dd-cfDNA over a time interval is indicative of transplant status. In some embodiments, the method further comprises adjusting immunosuppressive therapy based on the levels of dd-cfDNA over the time interval. In some embodiments, an increase in the levels of dd-cfDNA are indicative of transplant rejection and a need for adjusting immunosuppressive therapy. In some embodiments, a change or a decrease in the levels of dd-cfDNA indicates transplant tolerance or stability, and a need for adjusting immunosuppressive therapy.

In some embodiments, in the methods disclosed herein, the target loci that are amplified in amplicons of about 50-100 bp in length, or about 60-80 bp in length. In some embodiments, the amplicons are about 65 bp in length.

In some embodiments, the methods disclosed herein further comprise measuring an amount of transplant DNA and an amount of recipient DNA in the recipient blood sample.

In some embodiments, the methods disclosed herein do not comprise genotyping the transplant donor and the transplant recipient.

In some embodiments, the methods disclosed herein further comprise detecting the amplified target loci using a microarray.

In some embodiments, in the methods disclosed herein, the polymorphic loci and the non-polymorphic loci are amplified in a single reaction.

In some embodiments, in the methods disclosed herein, the DNA is preferentially enriched at the target loci.

In some embodiments, preferentially enriching the DNA in the sample at the plurality of polymorphic loci includes obtaining a plurality of pre-circularized probes where each probe targets one of the polymorphic loci, and where the 3′ and 5′ end of the probes are designed to hybridize to a region of DNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to DNA from the sample, filling the gap between the hybridized probe ends using DNA polymerase, circularizing the pre-circularized probe, and amplifying the circularized probe.

In some embodiments, preferentially enriching the DNA at the plurality of polymorphic loci includes obtaining a plurality of ligation-mediated PCR probes where each PCR probe targets one of the polymorphic loci, and where the upstream and downstream PCR probes are designed to hybridize to a region of DNA, on one strand of DNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the DNA from the first sample, filling the gap between the ligation-mediated PCR probe ends using DNA polymerase, ligating the ligation-mediated PCR probes, and amplifying the ligated ligation-mediated PCR probes.

In some embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some embodiments, preferentially enriching the DNA at a plurality of polymorphic loci includes obtaining a plurality of inner forward primers where each primer targets one of the polymorphic loci, and where the 3′ end of the inner forward primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′ end of the inner reverse primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the DNA, and amplifying the DNA using the polymerase chain reaction to form amplicons.

In some embodiments, the method also includes obtaining a plurality of outer forward primers where each primer targets one of the polymorphic loci, and where the outer forward primers are designed to hybridize to the region of DNA upstream from the inner forward primer, optionally obtaining a plurality of outer reverse primers where each primer targets one of the polymorphic loci, and where the outer reverse primers are designed to hybridize to the region of DNA immediately downstream from the inner reverse primer, hybridizing the first primers to the DNA, and amplifying the DNA using the polymerase chain reaction.

In some embodiments, the method also includes obtaining a plurality of outer reverse primers where each primer targets one of the polymorphic loci, and where the outer reverse primers are designed to hybridize to the region of DNA immediately downstream from the inner reverse primer, optionally obtaining a plurality of outer forward primers where each primer targets one of the polymorphic loci, and where the outer forward primers are designed to hybridize to the region of DNA upstream from the inner forward primer, hybridizing the first primers to the DNA, and amplifying the DNA using the polymerase chain reaction.

In some embodiments, preparing the first sample further includes appending universal adapters to the DNA in the first sample and amplifying the DNA in the first sample using the polymerase chain reaction. In some embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some embodiments, amplifying the DNA is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some embodiments, preparing the sample further comprises dividing the sample into a plurality of portions, and where the DNA in each portion is preferentially enriched at a subset of the plurality of polymorphic loci. In some embodiments, the inner primers are selected by identifying primer pairs likely to form undesired primer duplexes and removing from the plurality of primers at least one of the pair of primers identified as being likely to form undesired primer duplexes. In some embodiments, the inner primers contain a region that is designed to hybridize either upstream or downstream of the targeted polymorphic locus, and optionally contain a universal priming sequence designed to allow PCR amplification. In some embodiments, at least some of the primers additionally contain a random region that differs for each individual primer molecule. In some embodiments, at least some of the primers additionally contain a molecular barcode.

In some embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some embodiments, the method does not comprise using a microarray.

In some embodiments, the method comprises (a) performing multiplex polymerase chain reaction (PCR) on the cell free DNA sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some embodiments, the method does not comprise using a microarray.

In some embodiments, the method also includes obtaining genotypic data from one or both of the transplant donor and the transplant recipient. In some embodiments, obtaining genotypic data from one or both of the transplant donor and the transplant recipient includes preparing the DNA from the donor and the recipient where the preparing comprises preferentially enriching the DNA at the plurality of polymorphic loci to give prepared DNA, optionally amplifying the prepared DNA, and measuring the DNA in the prepared sample at the plurality of polymorphic loci.

In some embodiments, building a joint distribution model for the expected allele count probabilities of the plurality of polymorphic loci on the chromosome is done using the obtained genetic data from the one or both of the transplant donor and the transplant recipient. In some embodiments, the first sample has been isolated from transplant recipient plasma and where the obtaining genotypic data from the transplant recipient is done by estimating the recipient genotypic data from the DNA measurements made on the prepared sample.

In some embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some embodiments, the plurality of polymorphic loci are SNPs. In some embodiments, measuring the DNA in the prepared sample is done by sequencing.

In some embodiments, a diagnostic box is disclosed for helping to determine transplant status in a transplant recipient where the diagnostic box is capable of executing the preparing and measuring steps of the disclosed methods.

In some embodiments, the allele counts are probabilistic rather than binary. In some embodiments, measurements of the DNA in the prepared sample at the plurality of polymorphic loci are also used to determine whether or not the transplant has inherited one or a plurality of linked haplotypes.

In some embodiments, building a joint distribution model for allele count probabilities is done by using data about the probability of chromosomes crossing over at different locations in a chromosome to model dependence between polymorphic alleles on the chromosome. In some embodiments, building a joint distribution model for allele counts and the step of determining the relative probability of each hypothesis are done using a method that does not require the use of a reference chromosome.

In some embodiments, determining the relative probability of each hypothesis makes use of an estimated fraction of donor-derived cell-free DNA (dd-cfDNA) in the prepared sample. In some embodiments, the DNA measurements from the prepared sample used in calculating allele count probabilities and determining the relative probability of each hypothesis comprise primary genetic data. In some embodiments, selecting the transplant status corresponding to the hypothesis with the greatest probability is carried out using maximum likelihood estimates or maximum a posteriori estimates.

In some embodiments, calling the transplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some embodiments, a confidence estimate is calculated for the called transplant status. In some embodiments, the method also includes taking a clinical action based on the called transplant status.

In some embodiments, a report displaying a determined transplant status is generated using the method. In some embodiments, a kit is disclosed for determining a transplant status designed to be used with the methods disclosed herein, the kit including a plurality of inner forward primers and optionally the plurality of inner reverse primers, where each of the primers is designed to hybridize to the region of DNA immediately upstream and/or downstream from one of the polymorphic sites on the target chromosome, and optionally additional chromosomes, where the region of hybridization is separated from the polymorphic site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some embodiments, the methods disclosed herein comprise a selection step to select for shorter cfDNA.

In some embodiments, the methods disclosed herein comprise a universal application step to enrich for cfDNA.

In some embodiments, the determination that the amount of dd-cfDNA above a cutoff threshold is indicative of acute rejection of the transplant. Machine learning may be used to resolve rejection vs non-rejection.

In some embodiments, the cutoff threshold value is expressed as a percentage of dd-cfDNA (dd-cfDNA %) in the blood sample.

In some embodiments, the cutoff threshold value is expressed as an amount of dd-cfDNA per volume unit of the blood sample.

In some embodiments, the cutoff threshold value is expressed as an amount of dd-cfDNA per volume unit of the blood sample multiplied by body mass or blood volume of the transplant recipient.

In some embodiments, the cutoff threshold value takes into account the body mass or blood volume of the patient.

In some embodiments, the cutoff threshold value takes into account one or more of the followings: donor genome copies per volume of plasma, cell-free DNA yield per volume of plasma, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI, TTV), recipient and/or donor HLA alleles or epitope mismatches, Banff classification of renal allograft pathology, and for-cause vs surveillance or protocol biopsy.

In some embodiments, the cutoff threshold value is scaled according to the amount of total cfDNA in the blood sample.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled according to the amount of total cfDNA in the blood sample and a confidence interval of 95%.

### Multiplex Amplification

In some embodiments, the method comprises performing a multiplex amplification reaction to amplify a plurality of polymorphic loci in one reaction mixture before determining the sequences of the selectively enriched DNA.

In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 100; 200; 500; 1,000; 2,000; 5,000; 10,000; 20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to at least 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify cfDNA obtained from a blood, plasma, serum, solid tissue, or urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some embodiments, the sequences of the amplicons are determined by performing high-throughput sequencing.

The genetic data of the transplant recipient and/or of the transplant donor can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS ‘ s TRUE SINGLE MOLECULE SEQUENCING platform, HALCYON MOLECULAR’ s electron microscope sequencing method, or any other sequencing method. In some embodiments, the high throughput sequencing is performed on Illumina NextSeq®, followed by demultiplexing and mapping to the human reference genome. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

### Tracer DNA and Use Thereof

Tracer DNA for estimating the amount of total cfDNA in a sample is described in U.S. Prov. Appl. No. 63/031,879 filed May 29, 2020 and titled “Improved Methods for Detection of Donor Derived Cell-Free DNA”, which is incorporated herein by reference in its entirety. In some embodiments, the Tracer DNA comprises synthetic double-stranded DNA molecules. In some embodiments, the Tracer DNA comprises DNA molecules of non-human origin.

In some embodiments, the Tracer DNA comprises DNA molecules having a length of about 50-500 bp, or about 75-300 bp, or about 100-250 bp, or about 125-200 bp, or about 125 bp, or about 160 bp, or about 200 bp, or about 500-1,000 bp.

In some embodiments, the Tracer DNA comprises DNA molecules having the same or substantially the same length, such as a DNA molecule having a length of about 125 bp, or about 160 bp, or about 200 bp. In some embodiments, the Tracer DNA comprises DNA molecules having different lengths, such as a first DNA molecule having a length of about 125 bp, a second DNA molecule having a length of about 160 bp, and a third DNA molecule having a length of about 200 bp. In some embodiments, the DNA molecules having different lengths are used to determine size distribution of the cell-free DNA in the sample

In some embodiments, the Tracer DNA comprises a target sequence, wherein the target sequence comprises a barcode positioned between a pair of primer binding sites capable of binding to a pair of primers. In some embodiments, at least part of the Tracer DNA is designed based on an endogenous human SNP locus, by replacing an endogenous sequence containing the SNP locus with the barcode. During the mmPCR target enrichment step, the primer pair targeting the SNP locus can also amplify the portion of Tracer DNA containing the barcode.

In some embodiments, the barcode is an arbitrary barcode. In some embodiments, the barcode comprises reverse complement of a corresponding endogenous genome sequence capable of being amplified by the same primer pair.

In some embodiments, the target sequence within the Tracer DNA is flanked on one or both sides by endogenous genome sequences. In some embodiments, the target sequence within the Tracer DNA is flanked on one or both sides by non-endogenous sequences.

In some embodiments, the Tracer DNA comprises a plurality of target sequences. In some embodiments, the Tracer DNA comprises a first target sequence comprising a first barcode positioned between a first pair of primer binding sites capable of binding to a first pair of primers, and a second barcode positioned between a second pair of primer binding sites capable of binding to a second pair of primers. In some embodiments, the first and/or second target sequence is designed based on one or more endogenous human SNP loci, by replacing an endogenous sequence containing a SNP locus with a barcode. In some embodiments, the first and/or second barcode is an arbitrary barcode. In some embodiments, the first and/or second barcode comprises reverse complement of a corresponding endogenous genome sequence capable of being amplified by the first or second primer pair. In some embodiments, the first and/or second target sequence within the Tracer DNA is flanked on one or both sides by endogenous genome sequences. In some embodiments, the first and/or second target sequence within the Tracer DNA is flanked on one or both sides by non-endogenous sequences.

In some embodiments, the Tracer DNA comprises DNA molecules having the same or substantially the same sequence. In some embodiments, the Tracer DNA comprises DNA molecules having different sequences.

In some embodiments, the Tracer DNA comprises a first DNA comprising a first target sequence and a second DNA comprising a second target sequence. In some embodiments, the first target sequence and second target sequence have different barcodes positioned between the same primer binding sites. In some embodiments, the first target sequence and second target sequence have different barcodes positioned between the same primer binding sites, wherein the different barcodes have the same or substantially the same lengths. In some embodiments, the first target sequence and second target sequence have different barcodes positioned between the same primer binding sites, wherein the different barcodes have different lengths. In some embodiments, the first target sequence and second target sequence are designed based on different endogenous human SNP loci, and hence comprise different primer binding sites. In some embodiments, the amount of first DNA and the amount of the second DNA are the same or substantially the same in the Tracer DNA. In some embodiments, the amount of first DNA and the amount of the second DNA are different in the Tracer DNA.

### Determining Amount of Total Cell-Free DNA Using Tracer DNA

In certain embodiments, Tracer DNA can be used to improve accuracy and precision of the method described herein, help quantify over a wider input range, assess efficiency of different steps at different size ranges, and/or calculate fragment size-distribution of input material.

Some embodiments of the present invention relate to a method of quantifying the amount of total cell-free DNA in a biological sample, comprising: a) isolating cell-free DNA from the biological sample, wherein a first Tracer DNA is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA.

In some embodiments, the method comprises adding the first Tracer DNA to a whole blood sample before plasma extraction. In some embodiments, the method comprises adding the first Tracer DNA to a plasma sample after plasma extraction and before isolation of the cell-free DNA. In some embodiments, the method comprises adding the first Tracer DNA to a composition comprising the isolated cell-free DNA. In some embodiments, the method comprises ligating adaptors to the isolated cell-free DNA to obtain a composition comprising adaptor-ligated DNA, and adding the first Tracer DNA to the composition comprising adaptor-ligated DNA.

In some embodiments, the method further comprises adding a second Tracer DNA before the targeted amplification. In some embodiments, the method further comprises adding a second Tracer DNA after the targeted amplification.

In some embodiments, the amount of total cfDNA in the sample is estimated using the NOR of the Tracer DNA (identifiable by the barcode), the NOR of sample DNA, and the known amount of the Tracer DNA added to the plasma sample. In some embodiments, the ratio between the NOR of the Tracer DNA and the NOR of sample DNA is used to quantify the amount of total cell-free DNA. In some embodiments, the ratio between the NOR of the barcode and the NOR of the corresponding endogenous genome sequence is used to quantify the amount of total cell-free DNA. In some embodiments, this information along with the plasma volume can also be used to calculate the amount of cfDNA per volume of plasma. In some embodiments, these can be multiplied by the percentage of donor DNA to calculate the total donor cfDNA and the donor cfDNA per volume of plasma.

Accordingly, in another aspect, the present invention relates to a method of quantifying the amount of total cell-free DNA in a biological sample, comprising: a) isolating cell-free DNA from the biological sample, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA composition.

In further aspect, the present invention relates to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

In a further aspect, the present invention relates to a method of determining the occurrence or likely occurrence of transplant rejection or graft injury, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of total cell-free DNA is quantified using sequencing reads derived from the first Tracer DNA composition, and determining the occurrence or likely occurrence of transplant rejection or graft injury using the amount of donor-derived cell-free DNA by comparing the amount of donor-derived cell-free DNA to a threshold value, wherein the threshold value is determined according to the amount of total cell-free DNA.

In some embodiments, the threshold value is a function of the number of sequencing reads of the donor-derived cell-free DNA.

In some embodiments, the method further comprises flagging the sample if the amount of total cell-free DNA falls outside a pre-determined range. In some embodiments, the method further comprises flagging the sample if the amount of total cell-free DNA is above a pre-determined value. In some embodiments, the method further comprises flagging the sample if the amount of total cell-free DNA is below a pre-determined value.

In some embodiments, the method comprises adding the first Tracer DNA composition to a whole blood sample before plasma extraction. In some embodiments, the method comprises adding the first Tracer DNA composition to a plasma sample after plasma extraction and before isolation of the cell-free DNA. In some embodiments, the method comprises adding the first Tracer DNA composition to a composition comprising the isolated cell-free DNA. In some embodiments, the method comprises ligating adaptors to the isolated cell-free DNA to obtain a composition comprising adaptor-ligated DNA, and adding the first Tracer DNA composition to the composition comprising adaptor-ligated DNA.

In some embodiments, the method further comprises adding a second Tracer DNA composition before the targeted amplification. In some embodiments, the method further comprises adding a second Tracer DNA composition after the targeted amplification.

In some embodiments, the first and/or second Tracer DNA composition comprises a plurality of DNA molecules having different sequences.

In some embodiments, the first and/or second Tracer DNA composition comprises a plurality of DNA molecules having at different concentrations.

In some embodiments, the first and/or second Tracer DNA composition comprises a plurality of DNA molecules having different lengths. In some embodiments, the plurality of DNA molecules having different lengths are used to determine size distribution of the cell-free DNA in the sample.

In some embodiments, the first and/or second Tracer DNA composition comprises a plurality of DNA molecules of non-human origin.

In some embodiments, the first and/or second Tracer DNA composition each comprises a target sequence, wherein the target sequence comprises a barcode positioned between a pair of primer binding sites capable of binding to one of the primer pairs. In some embodiments, the barcode comprises reverse complement of a corresponding endogenous genome sequence capable of being amplified by the same primer pair.

In some embodiments, the ratio between the number of reads of the Tracer DNA and the number of reads of sample DNA is used to quantify the amount of total cell-free DNA. In some embodiments, the ratio between the number of reads of the barcode and the number of reads of the corresponding endogenous genome sequence is used to quantify the amount of total cell-free DNA.

In some embodiments, the target sequence is flanked on one or both sides by endogenous genome sequences. In some embodiments, the target sequence is flanked on one or both sides by non-endogenous sequences.

In some embodiments, the first and/or second Tracer DNA composition comprises synthetic double-stranded DNA molecules. In some embodiments, the first and/or second Tracer DNA composition comprises DNA molecules having a length of 50-500 bp. In some embodiments, the first and/or second Tracer DNA composition comprises DNA molecules having a length of 75-300 bp. In some embodiments, the first and/or second Tracer DNA composition comprises DNA molecules having a length of 100-250 bp. In some embodiments, the first and/or second Tracer DNA composition comprises DNA molecules having a length of 125-200 bp. In some embodiments, the first and/or second Tracer DNA composition comprises DNA molecules having a length of about 200 bp. In some embodiments, the first and/or second Tracer DNA composition comprises DNA molecules having a length of about 160 bp. In some embodiments, the first and/or second Tracer DNA composition comprises DNA molecules having a length of about 125 bp. In some embodiments, the first and/or second Tracer DNA composition comprises DNA molecules having a length of 500-1,000 bp.

In some embodiments, the targeted amplification comprises amplifying at least 100 polymorphic or SNP loci in a single reaction volume. In some embodiments, the targeted amplification comprises amplifying at least 200 polymorphic or SNP loci in a single reaction volume. In some embodiments, the targeted amplification comprises amplifying at least 500 polymorphic or SNP loci in a single reaction volume. In some embodiments, the targeted amplification comprises amplifying at least 1,000 polymorphic or SNP loci in a single reaction volume. In some embodiments, the targeted amplification comprises amplifying at least 2,000 polymorphic or SNP loci in a single reaction volume. In some embodiments, the targeted amplification comprises amplifying at least 5,000 polymorphic or SNP loci in a single reaction volume. In some embodiments, the targeted amplification comprises amplifying at least 10,000 polymorphic or SNP loci in a single reaction volume.

In some embodiments, each primer pair is designed to amplify a target sequence of about 35 to 200 bp. In some embodiments, each primer pair is designed to amplify a target sequence of about 50 to 100 bp. In some embodiments, each primer pair is designed to amplify a target sequence of about 60 to 75 bp. In some embodiments, each primer pair is designed to amplify a target sequence of about 65 bp.

In some embodiments, the transplant recipient is a human subject. In some embodiments, the transplant is a human transplant. In some embodiments, the transplant is a pig transplant. In some embodiments, the transplant is from a non-human animal.

In some embodiments, the transplant is an organ transplant, tissue transplant, or cell transplant. In some embodiments, the transplant is a kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, or blood transfusion.

In some embodiments, the method further comprises determine the transplant rejection as antibody mediated transplant rejection, T-cell mediated transplant rejection, graft injury, viral infection, bacterial infection, or borderline rejection. In some embodiments, the method further comprises determining the likelihood of one or more cancers. Cancer screening, detection, and monitoring are disclosed in PCT Patent Publication Nos. WO2015/164432, WO2017/181202, WO2018/083467, and WO2019/200228, each of which is incorporated herein by reference in its entirety. In other embodiments, the invention relates to screening a patient to determine their predicted responsiveness, or resistance, to one or more cancer treatments. This determination can be made by determining the existence of wild-type vs. mutated forms of a target gene, or in some cases the increased or over-expression of a target gene. Examples of such target screens include KRAS, NRAS, EGFR, ALK, KIT, and others. For example, a variety of KRAS mutations are appropriate for screening in accordance with the invention including, but not limited to, G12C, G12D, G12V, G13C, G13D, A 18D, Q61H, K117N. In addition, PCT Patent Publication Nos. WO2015/164432, WO2017/181202, WO2018/083467, and WO2019/200228, which are incorporated herein by reference in their entirety.

In some embodiments, the method is performed without prior knowledge of donor genotypes. In some embodiments, the method is performed without prior knowledge of recipient genotypes. In some embodiments, the method is performed without prior knowledge of donor and/or recipient genotypes. In some embodiments, no genotyping of either the donor or the recipient is required prior to performing the method.

In some embodiments, the biological sample is a blood sample. In some embodiments, the biological sample is a plasma sample. In some embodiments, the biological sample is a serum sample. In some embodiments, the biological sample is a urine sample. In some embodiments, the biological sample is a sample of lymphatic fluid. In some embodiments, the sample is a solid tissue sample.

In some embodiments, the method further comprises longitudinally collecting a plurality of biological samples from the transplant recipient, and repeating steps (a) to (d) for each sample collected.

In some embodiments, the quantifying step comprises determining the percentage of donor-derived cell-free DNA out of the total of donor-derived cell-free DNA and recipient-derived cell-free DNA in the blood sample. In some embodiments, the quantifying step comprises determining the amount of donor-derived cell-free DNA. In some embodiments, the quantifying step comprises determining the amount of donor-derived cell-free DNA per volume unit of the blood sample.

In another aspect, the present invention relates to a method of diagnosing a transplant within a transplant recipient as undergoing acute rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of donor-derived cell-free DNA above a threshold value indicates that the transplant is undergoing acute rejection, wherein the threshold value is determined according to the amount of total cell-free DNA, and wherein the amount of total cell-free DNA is quantified using sequencing reads derived from the first Tracer DNA composition.

In another aspect, the present invention relates to a method of monitoring immunosuppressive therapy in a transplant recipient, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein a change in levels of donor-derived cell-free DNA over a time interval is indicative of transplant status, wherein the levels of donor-derived cell-free DNA is scaled according to the amount of total cell-free DNA, and wherein the amount of total cell-free DNA is quantified using sequencing reads derived from the first Tracer DNA composition.

In some embodiments, the method further comprises adjusting immunosuppressive therapy based on the levels of dd-cfDNA over the time interval.

In some embodiments, an increase in the levels of dd-cfDNA is indicative of transplant rejection or graft injury and a need for adjusting immunosuppressive therapy. In some embodiments, no change or a decrease in the levels of dd-cfDNA indicates transplant tolerance or stability, and a need for adjusting immunosuppressive therapy.

In some embodiments, the method further comprises size selection to enrich for donor-derived cell-free DNA and reduce the amount of recipient-derived cell-free DNA disposed from bursting white-blood cells.

In some embodiments, the method further comprises a universal amplification step that preferentially amplifies donor-derived cell-free DNA over recipient-derived cell-free DNA originating from lysed or damaged white-blood cells.

In some embodiments, the method comprises longitudinally collecting a plurality of blood, plasma, serum, solid tissue, or urine samples from the transplant recipient after transplantation, and repeating steps (a) to (d) for each sample collected. In some embodiments, the method comprises collecting and analyzing blood, plasma, serum, solid tissue, or urine samples from the transplant recipient for a time period of about three months, or about six months, or about twelve months, or about eighteen months, or about twenty-four months, etc. In some embodiments, the method comprises collecting blood, plasma, serum, solid tissue, or urine samples from the transplant recipient at an interval of about one week, or about two weeks, or about three weeks, or about one month, or about two months, or about three months, etc.

In some embodiments, the determination that the amount of dd-cfDNA above a cutoff threshold is indicative of acute rejection of the transplant. Machine learning may be used to resolve rejection vs non-rejection. Machine learning is disclosed in WO2020/018522, titled “Methods and Systems for calling Ploidy States using a Neural Network” and filed on Jul. 16, 2019 as PCT/US2019/041981, which is incorporated herein by reference in its entirety. In some embodiments, the cutoff threshold value is scaled according to the amount of total cfDNA in the blood sample.

In some embodiments, the cutoff threshold value is expressed as percentage of dd-cfDNA (dd-cfDNA %) in the blood sample. In some embodiments, the cutoff threshold value is expressed as quantity or absolute quantity of dd-cfDNA. In some embodiments, the cutoff threshold value is expressed as quantity or absolute quantity of dd-cfDNA per volume unit of the blood sample. In some embodiments, the cutoff threshold value is expressed as quantity or absolute quantity of dd-cfDNA per volume unit of the blood sample multiplied by body mass, BMI, or blood volume of the transplant recipient.

In some embodiments, the cutoff threshold value takes into account the body mass, BMI, or blood volume of the patient. In some embodiments, the cutoff threshold value takes into account one or more of the following: donor genome copies per volume of plasma, cell-free DNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some embodiments, the method has a sensitivity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled or adjusted according to the amount of total cfDNA in the blood sample and a confidence interval of 95%.

In some embodiments, the method has a specificity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled or adjusted according to the amount of total cfDNA in the blood sample and a confidence interval of 95%.

### Adjusting Threshold for Calling Transplant Rejection or Graft Injury Using Amount of Total Cell-Free DNA

Some embodiments of the present invention relate to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

Some embodiments use either a fixed threshold of donor DNA per plasma volume or one that is not fixed, such as adjusted or scaled as noted herein. The way that this is determined can be based on using a training data set to build an algorithm to maximize performance. It may also take into account other data such as patient weight, age, or other clinical factors.

In some embodiments, the method further comprises determining the occurrence or likely occurrence of transplant rejection or graft injury using the amount of donor-derived cell-free DNA. In some embodiments, the amount of donor-derived cell-free DNA is compared to a cutoff threshold value to determine the occurrence or likely occurrence of transplant rejection or graft injury, wherein the cutoff threshold value is adjusted or scaled according to the amount of total cell-free DNA. In some embodiments, the cutoff threshold value is a function of the number of reads of the donor-derived cell-free DNA.

In some embodiments, the method comprises applying a scaled or dynamic threshold metric that takes into account the amount of total cfDNA in the samples to more accurately assess transplant rejection or graft injury. In some embodiments, the method further comprises flagging the sample if the amount of total cell-free DNA is above a pre-determined value. In some embodiments, the method further comprises flagging the sample if the amount of total cell-free DNA is below a pre-determined value.

### WORKING EXAMPLES

### Example 1

The workflow of this non-limiting example corresponds to the workflow disclosed in Sigdel et al., J. Clin. Med. 8(1):19 (2019), which is incorporated herein by reference in its entirety. This example is illustrative only, and a skilled artisan will appreciate that the invention disclosed herein can be practiced in a variety of other ways.

Blood Samples

Male and female adult or young-adult patients received a kidney from related or unrelated living donors, or unrelated deceased donors. Time points of patient blood draw following transplantation surgery were either at the time of an allograft biopsy or at various pre-specified time intervals based on lab protocols. Typically, samples were biopsy-matched and had blood drawn at the time of clinical dysfunction and biopsy or at the time of protocol biopsy (at which time most patients did not have clinical dysfunction). In addition, some patients had serial post transplantation blood drawn. The selection of study samples was based on (a) adequate plasma being available, and (b) if the sample was associated with biopsy information. Among the full 300 sample cohort, 72.3% were drawn on the day of biopsy.

Dd-cfDNA Measurement in Blood Samples

Cell-free DNA was extracted from plasma samples using the QIAamp Circulating Nucleic Acid Kit (Qiagen) and quantified on the LabChip NGS 5k kit (Perkin Elmer, Waltham, MA, USA) following manufacturer's instructions. Cell-free DNA was input into library preparation using the Natera Library Prep kit as described in Abbosh et al, Nature 545: 446-451 (2017), with a modification of 18 cycles of library amplification to plateau the libraries. Purified libraries were quantified using LabChip NGS 5k as described in Abbosh et al, Nature 545: 446-451 (2017). Target enrichment was accomplished using massively multiplexed-PCR (mmPCR) using a modified version of a described in Zimmermann et al., Prenat. Diagn. 32:1233-1241 (2012), with 13,392 single nucleotide polymorphisms (SNPs) targeted. Amplicons were then sequenced on an Illumina HiSeq 2500 Rapid Run®, 50 cycles single end, with 10-11 million reads per sample.

Statistical Analyses of dd-cfDNA and eGFR

In each sample, dd-cfDNA was measured and correlated with rejection status, and results were compared with eGFR. Where applicable, all statistical tests were two sided. Significance was set at p<0.05. Because the distribution of dd-cfDNA in patients was severely skewed among the groups, data were analyzed using a Kruskal-Wallis rank sum test followed by Dunn multiple comparison tests with Holm correction. eGFR (serum creatinine in mg/dL) was calculated as described previously for adult and pediatric patients. Briefly, eGFR=186×Serum Creatinine^{-1.154}×Age−^{-0.203}×(1.210 if Black)×(0.742 if Female).

To evaluate the performance of dd-cfDNA and eGFR (mL/min/1.73 m²) as rejection markers, samples were separated into an AR group and a non-rejection group (BL+STA+OI). Using this categorization, the following predetermined cut-offs were used to classify a sample as AR: >1% for dd-cfDNA and <60.0 for eGFR.

To calculate the performance parameters of each marker (sensitivity, specificity, positive predictive value (PPV), negative predictive value (NPV), and area under the curve (AUC)), a bootstrap method was used to account for repeated measurements within a patient. Briefly, at each bootstrap step, a single sample was selected from each patient; by assuming independence among patients, the performance parameters and their standard errors were calculated. This was repeated 10,000 times; final confidence intervals were calculated using the bootstrap mean for the parameter with the average of the bootstrap standard errors with standard normal quantiles. Standard errors for sensitivity and specificity were calculated assuming a binomial distribution; for PPV and NPV a normal approximation was used; and for AUC the DeLong method was used. Performance was calculated for all samples with a matched biopsy, including the sub-cohort consisting of samples drawn at the same time as a protocol biopsy.

Differences in dd-cfDNA levels by donor type (living related, living non-related, and deceased non-related) were also evaluated. Significance was determined using the Kruskal-Wallis rank sum test as described above. Inter- and intra-variability in dd-cfDNA over time was evaluated using a mixed effects model with a logarithmic transformation on dd-cfDNA; 95% confidence intervals (CI) for the intra- and inter-patient standard deviations were calculated using a likelihood profile method.

Post hoc analyses evaluated (a) different dd-cfDNA thresholds to maximize NPV and (b) combined dd-cfDNA and eGFR to define an empirical rejection zone that may improve the PPV for AR diagnosis. All analyses were done using R 3.3.2 using the FSA (for Dunn tests), lme4 (for mixed effect modeling) and pROC (for AUC calculations) packages.

Biopsy Samples

Optionally, kidney biopsies were analyzed in a blinded manner by a pathologist and were graded by the 2017 Banff classification for active rejection (AR); intragraft C4d stains were performed to assess for acute humoral rejection. Biopsies were not done in cases of active urinary tract infection (UTI) or other infections. Transplant “injury” was defined as a >20% increase in serum creatinine from its previous steady-state baseline value and an associated biopsy that was classified as either active rejection (AR), borderline rejection (BL), or other injury (OI) (e.g., drug toxicity, viral infection). Active rejection was defined, at minimum, by the following criteria: (1) T-cell-mediated rejection (TCMR) consisting of either a tubulitis (t) score >2 accompanied by an interstitial inflammation (i) score >2 or vascular changes (v) score >0; (2) C4d positive antibody-mediated rejection (ABMR) consisting of positive donor specific antibodies (DSA) with a glomerulitis (g) score >0/or peritubular capillaritis score (ptc)>0 or v>0 with unexplained acute tubular necrosis/thrombotic micro angiopathy (ATN/TMA) with C4d=2; or (3) C4d negative ABMR consisting of positive DSA with unexplained ATN/TMA with g+ptc≥2 and C4d is either 0 or 1. Borderline change (BL) was defined by t1+i0, or t1+i1, or t2+i0 without explained cause (e.g., polyomavirus-associated nephropathy (PVAN)/infectious cause/ATN). Other criteria used for BL changes were g>0 and/or ptc>0, or v>0 without DSA, or C4d or positive DSA, or positive C4d without nonzero g or ptc scores. Normal (STA) allografts were defined by an absence of significant injury pathology as defined by Banff schema.

### Example 2

This example is illustrative only, and a skilled artisan will appreciate that the invention disclosed herein can be practiced in a variety of other ways. The workflow described in Example 1 is modified by adding one or more Tracer DNA(s) each containing a SNP locus to the plasma sample prior to extraction of cell-free DNA, as described in U.S. Prov. Appl. No. 63/031,879 filed May 29, 2020 and titled “Improved Methods for Detection of Donor Derived Cell-Free DNA”, which is incorporated herein by reference in its entirety. During the mmPCR target enrichment step, the primer pairs targeting the SNP locus also amplify the Tracer DNA(s). The amount of total cfDNA in the sample is estimated using the number of sequences reads of the Tracer DNA(s) which are identifiable by the barcode, the number of sequences reads of sample DNA, and the known amount of the Tracer DNA(s) added to the plasma sample.

### Example 3

This example is illustrative only, and a skilled artisan will appreciate that the invention disclosed herein can be practiced in a variety of other ways. The workflow described in Example 1 is used to process and analyze plasma samples from simultaneous pancreas-kidney transplant (SPK) recipients and sequential pancreas after kidney transplant (PAK) recipients. Cutoff thresholds of 1% dd-cfDNA or 1.5% dd-cfDNA successfully identified SPK transplant recipients having acute rejection from transplant recipients with stable graft.

### Example 4

This example is illustrative only, and a skilled artisan will appreciate that the invention disclosed herein can be practiced in a variety of other ways.

Early detection of allograft rejection is critical to the successful management of transplant recipients. Tissue biopsy has been the “gold standard” for diagnosis of active rejection (AR), but is invasive and has poor reproducibility. Conventional non-invasive biomarkers such as changes in serum creatinine are available for detecting AR, but are limited due to low sensitivity and specificity. Thus, there is a need for new non-invasive markers that have high accuracy for detecting AR.

Donor-derived cell-free DNA fraction (dd-cfDNA (%)) is a promising non-invasive biomarker for detecting allograft rejection. However, dd-cfDNA (%) can be artificially depressed by high levels of circulating cfDNA, which can occur in patients who are obese, have had recent surgery, medical complications, or received certain medications. This can potentially lead to false negative results.

Recently, two studies provided preliminary evidence indicating that the absolute quantity of dd-cfDNA may show better performance for detecting AR than dd-cfDNA (%). Here we present results from an assay that utilizes a new two-threshold algorithm which combines both dd-cfDNA (%) and absolute quantity of dd-cfDNA (copies/mL) with the goal of increasing test sensitivity, particularly through improved detection in cases where cfDNA levels are high.

The study included 41 patients undergoing allograft management, who received dd-cfDNA testing as part of routine clinical care. Patients who were under 18, pregnant, had an organ transplant other than kidney or a blood transfusion within 2 weeks of enrollment, were excluded.

Laboratory testing was performed by amplifying cfDNA using massively multiplexed-PCR (mmPCR), targeting over 13,000 single nucleotide polymorphisms. The dd-cfDNA fraction was measured according to Altug et al., Transplantation, 103:2657-2665 (2019); the absolute concentration of dd-cfDNA was calibrated to give the quantity of dd-cfDNA (cp/mL). The new two-threshold algorithm combined the previously validated dd-cfDNA fraction cut-off (>1% indicating at-risk for rejection) and a previously established dd-cfDNA quantity cut-off of >78 cp/mL. Samples exceeding either threshold were considered at high risk for AR.

Matched biopsy results (for cause biopsies occurring within 4 weeks of dd-cfDNA testing) were available for 16 patients; 14/16 occurred within 2 weeks of the dd-cfDNA test. Biopsy samples were analyzed and graded by pathologists according to standard practice using Banff 2017 classification. Samples without biopsy were classified as not having active rejection based on clinical assessment (stable according to serum creatinine and other clinical indicators). AR was found in 9 of 16 (56%) biopsies performed, with 5 classified as T-cell mediated rejection (TCMR), 1 as antibody mediated rejection (ABMR) and 3 as mixed type (ABMR/TCMR).

We calculated sensitivity and specificity for each algorithm for the 41 patients in the sample. The original method, based on the >1% dd-cfDNA cutoff had a sensitivity of 7/9 (77.8%; 95% CI: 40.0-97.2%) and a specificity of 29/32 (90.6%, 95% CI: 75.0-98.0%). Applying the two-threshold algorithm to the data set, yielded a sensitivity of 9/9 (100%, 95% CI: 66.4-100%), and a specificity of 28/32 (87.5%; 95% CI: 71.0-96.5%).

Our results suggest that using both dd-cfDNA quantity and dd-cfDNA fraction to assess the rejection status of allograft can improve performance over just using dd-cfDNA fraction alone. Consistent with expectations, the three patients whose calls changed with the introduction of the new threshold had high total cfDNA levels, and thus depressed donor fractions that led, in two of the cases, to false negative results when using the 1% donor fraction cut off alone.

In conclusion, this study suggests that the combination of the quantity of dd-cfDNA threshold and the previously validated dd-cfDNA (%) threshold can produce improved sensitivity in the detection of AR in renal allograft patients while maintaining high specificity.

### Example 5

This example is illustrative only, and a skilled artisan will appreciate that the invention disclosed herein can be practiced in a variety of other ways.

Background: Donor-derived cell-free DNA (dd-cfDNA) fraction and quantity have both been shown to be associated with allograft rejection. The present study examined the relative predictive power of each of these variables to the combination of the two developed an algorithm incorporating both variables to detect active rejection in renal allograft biopsies.

Methods: The first 426 sequential indication biopsy samples collected with microarray-derived gene expression and dd-cfDNA results were included. After exclusions to simulate intended clinical use, 367 samples were analyzed. Biopsies were assessed using the Molecular Microscope Diagnostic System (MMDx) and histology (Banff 2019). Logistic regression analysis examined whether combining dd-cfDNA fraction and quantity adds predictive value to either alone. The first 149 sequential samples were used to develop a two-threshold algorithm, and the next 218 to validate the algorithm.

Results: In regression, the combination of dd-cfDNA fraction and quantity was found to be significantly more predictive than either variable alone (p-value 0.009 and <0.0001). In the test set, the AUC of the two-variable system was 0.88 and performance of the two-threshold algorithm showed sensitivity 83.1% and specificity 81.0% for molecular diagnoses, and sensitivity 73.5% and specificity 80.8% for histology diagnoses.

Conclusions: This prospective, biopsy-matched, multi-site dd-cfDNA study in kidney transplant patients found that the combination of dd-cfDNA fraction and quantity was more powerful than either dd-cfDNA fraction or quantity alone, and validated a novel two-threshold algorithm incorporating both variables.

### Example 6

This example is illustrative only, and a skilled artisan will appreciate that the invention disclosed herein can be practiced in a variety of other ways.

Background: Pancreas graft status in simultaneous pancreas kidney transplant (SPKTx) is currently assessed by non-specific biochemical markers, typically amylase and/or lipase. Identifying a non-invasive biomarker with good sensitivity in detecting early pancreas graft rejection could improve SPKTx management.

Methods: Here, we developed a pilot study to explore the performance of donor derived cell-free DNA (dd-cfDNA) in predicting biopsy-proven acute rejection of the pancreas graft in a cohort of SPKTx recipients. We used the Prospera™ test (Natera, Inc.) to measure dd-cfDNA in 36 SPKTx recipients who had at least one biopsy-matched plasma samples. Dd-cfDNA was reported both as a fraction of the total cfDNA (fraction; %) and as concentration in the recipient's plasma (quantity; copies/mL).

Results: In the absence of pancreas biopsy-proven acute rejection (P-BPAR) dd-cfDNA was significantly higher in samples collected within the first 45 days after SPKTx compared to those measured afterwards (median (%): 1.00 vs 0.30, median (cp/mL) 128.2 vs 53.3, respectively, p=0.001). In samples obtained beyond day 45, P-BPAR samples presented a significantly higher dd-cfDNA fraction (0.83 vs 0.30%, p=0.006) and quantity (81.3 vs 53.3 cp/mL; p=0.001) than stable samples. Incorporating dd-cfDNA quantity along with dd-cfDNA fraction outperformed dd-cfDNA fraction alone to detect active rejection. Notably, when using a quantity cut off of 70 cp/mL, dd-cfDNA detected P-BPAR with a sensitivity of 85.7% and a specificity of 93.7% for the diagnosis of P-BPAR, which was more accurate than current biomarkers (AUC of 0.89 for dd-cfDNA compared to 0.74 of lipase and 0.46 for amylase).

Conclusions: Dd-cfDNA measurement through a simple noninvasive blood test could be incorporated into clinical practice to help inform graft management in SPKTx patients.

### Example 7

This example is illustrative only, and a skilled artisan will appreciate that the invention disclosed herein can be practiced in a variety of other ways.

Simultaneous Pancreas Kidney transplant (SPKTx) is considered the best treatment alternative for patients with Type 1 diabetes (T1D) and end stage renal disease (ESRD). Diabetic nephropathy is a microvascular complication caused by sustained hyperglycemia and is one of the leading causes of ESRD. SPKTx can significantly improve prognosis and health status in patients with insulin-dependent diabetes as it can re-establish euglycemia and thus lead to a reduction of the predicted risk for of micro- and macrovascular complications.

Pancreas graft rejection is a leading cause of graft failure, with acute rejection incidences of up to 21% in the first 12 months. Current tools for assessing graft rejection rely on clinical laboratory tests that evaluate the exocrine (e.g., amylase, lipase) or endocrine (e.g. glycemia, HbA1C, C-Peptide) functionality of the graft. Remarkably, these tests are highly unspecific since the native pancreas' exocrine function is preserved in most patients, and hence elevation in any of these parameters may not be related to pancreas graft rejection. Pancreas graft biopsy is the gold standard for the diagnosis of acute rejection. However, biopsies are an invasive procedure with a significant rate of complications, and often cannot be performed or provide no significant information (up to 39% of the time) despite presence of graft dysfunction. Therefore, a clear need exists for a non-invasive, donor-specific, dynamic biomarker to assess allograft status and monitor for injury/rejection that can ultimately improve management in SPKTx transplant recipients.

Several studies have demonstrated that measurement of donor derived cell-free DNA (dd-cfDNA) in the blood of recipients of solid organ transplants (lung, kidney, heart, liver) can distinguish the risk of allograft rejection from non-rejection. Studies evaluating the potential of dd-cfDNA for assessing risk of rejection in SPKTx transplant recipients are limited. In this pilot study, we evaluated the performance of the Prospera™ test, which uses a SNP-based mmPCR methodology to measure dd-cfDNA both fraction and quantity, to assess risk of rejection in 36 SPK transplant recipients who were histologically profiled for graft status.

Materials and Methods

Study Design and patient's population. Pancreas biopsy-paired plasma samples (n=41) were included in the analysis. To account for the possible influence of donor-related and immediate post-operative complications on dd-cfDNA quantification we considered samples collected prior to and after 45 days after SPK transplantation separately. Out of the total 41 graft biopsies, 18 were collected <45 days post-transplant and 23 were collected >45 days post-transplant.

Patient's samples. Pancreas graft biopsies were performed either per protocol or for-cause. As per center protocol, for cause biopsies were indicated if patients presented a persistent (>2 determinations separated >48 h apart) elevation (>2× normal value) in pancreatic enzymes (amylase and/or lipase). Samples were obtained by ultrasound guided percutaneous needle punch and classified according to the 2011 Banff criteria. For analysis purposes, biopsies were further reclassified as ‘no-rejection’ or rejection, the latter including Banff categories: indeterminate, T-cell mediated rejection (TCMR), and antibody mediated rejection (ABMR). Whole blood and serum samples were obtained on the day of pancreas graft biopsy, prior to the performance of biopsy, to avoid misleading interpretation of dd-cfDNA. Whole blood samples were used to measure dd-cfDNA levels, whereas serum samples were used to measure amylase (U/1), lipase (U/1), creatinine (mg/dL), and anti-glutamic acid decarboxylase antibodies (GAD). In addition, serum samples were screened for HLA class I and II donor specific antibodies (DSAs) using the Lifecodes LifeScreen Deluxe flow bead assay (Immucor, Stamford, CT, USA). Antibody specificities were determined using the Lifecodes Single Antigen bead assay (Immucor, Stamford, CT, USA) in patients with positive screening for HLA antibodies. The DSAs were considered positive with mean fluorescence intensity (MFI) greater than 1500 according to the protocols of the Histocompatibility Laboratory of Catalunya. A/B/DRB1 HLA loci were considered for DSA in all patients, whereas DQB1/DP1/C HLA loci were considered for DSA only when there were available.

Assessment of dd-cfDNA levels using the Prospera™ test. Whole blood was drawn into PAXgene blood ccfDNA DNA tubes (QIAGEN®), and plasma samples were obtained through double centrifugation of whole blood following the manufacturer's instructions. Plasma was then stored at −80 C until sample processing. Massively multiplexed-PCR (mmPCR) was used to amplify cfDNA in plasma samples, targeting 13,926 SNPs, followed by sequencing of amplicons (Prospera™, Natera Inc., Austin, TX) as described in Altug et al., Transplantation, 103:2657-2665 (2019. Samples were run according to standard CLIA protocol, except for samples with <4 mL plasma which had 9 additional PCR cycles. Dd-cfDNA levels were reported as a fraction of the total cfDNA (%; median [IQR]) and as a concentration in the recipient's plasma (copies/mL; median [IQR]).

Statistical analysis. Comparisons of median measurements were performed using Mann-Whitney U test and P value <0.05 was considered statistically significant. When needed, P values were adjusted for multiple testing using Benjamini-Hochberg (BH) adjustment. ROC curves were constructed and sensitivity, specificity, positive predictive value (PPV), and negative predictive value (NPV) were calculated for various thresholds. Statistical analyses were performed in Python programming language using SciPy and statsmodels packages (Python Software Foundation, version, (https://www.python.org/psf/). Graphical representation of continuous variables is shown as median and Inter-Quartile Range [IQR].

Dd-cfDNA and pancreas graft rejection. The median dd-cfDNA fraction was significantly higher in patients with biopsy-proven acute rejection of pancreatic graft (P-BPAR; 1.05% [0.81-1.67]): compared to those with non-rejection (0.52% [IQR: 0.21-0.78]), p=0.0004). Similarly, the median absolute dd-cfDNA quantity was significantly higher in patients with P-BPAR (103.70 cp/mL [IQR: 76.70-189.80]) compared to those with non-rejection (51.5 cp/mL [IQR: 22.2-76.7]; p=0.0007). These data suggest that both dd-cfDNA fraction and quantity can discriminate between pancreatic graft rejection and non-rejection status in SPKTx recipients.

To explore the potential confounding factor of donor and surgery-associated organ injury we compared the dd-cfDNA levels before and after 45 days post-transplant. In patients with no rejection, the fraction of dd-cfDNA and absolute quantity of dd-cfDNA were significantly higher in the early post-op period compared to those with biopsy performed after day 45 (median % 1.00 vs 0.30, respectively, p=0.001; median cp/mL 128.2 vs 35.3, respectively, p=0.001). During the first 45 days after SPKTx, there were no statistical differences in dd-cfDNA levels between non-rejection samples and those with BPAR, either as a fraction of dd-cfDNA (p=0.120) or as dd-cfDNA quantity (p=0.290). In contrast, in biopsy-matched blood samples collected >45 days post-transplant, both dd-cfDNA fraction and dd-cfDNA quantity were significantly higher in the BPAR cohort (0.83% [IQR 0.67-1.58]; 81.3 cp/mL[IQR 73.4-152.0]) compared to the non-rejection cohort (0.30% [IQR 0.14-0.52], p=0.006, and 35.3 cp/mL [IQR 19.5-55.0], p=0.001, respectively). When excluding indeterminate biopsies from the acute rejection group, dd-cfDNA levels were still significantly elevated compared to non-rejection cases (0.81% [0.52-0.83] rejection vs 0.30% [0.14-0.52] non-rejection, p=0.031). These data suggest that both dd-cfDNA fraction and quantity can distinguish between graft rejection and non-rejection status after 45 days post-transplant.

We next aimed to identify an optimum cut-off value of dd-cfDNA that would accurately discriminate pancreatic graft rejection from non-rejection. We assessed the ability of two recently published thresholds in detecting kidney allograft rejection by applying a) a cut-off of 1% dd-cfDNA, and b) a two-threshold algorithm which combined the dd-cfDNA fraction cut-off (>1%) and a dd-cfDNA quantity cut-off of >78 cp/mL. Sensitivity using dd-cfDNA fraction alone was 28.6% ( 2/7). Sensitivity was considerably higher, at 57.1% ( 4/7), when using the two-threshold algorithm that combines dd-cfDNA fraction and dd-cfDNA quantity. Specificity was excellent for both cut-offs, at 100% and 93.7%, respectively. When using a dd-cfDNA quantity cut-off value of 70 cp/mL, the sensitivity increased to 85.7% ( 6/7) while maintaining a high specificity of 93.7%, along with a PPV of 85.7% and NPV of 93.7%.

Dd-cfDNA and DSA. Although only one of the biopsies collected >45 days post-transplant was characterized with antibody mediated rejection (ABMR), three patients were found to have circulating DSA. We found that dd-cfDNA fraction was significantly elevated in samples tested positive for DSA (0.83% (0.82-2.5)) vs those tested negative (0.39% [0.18-0.55]; p=0.022). Similarly, we found that dd-cfDNA quantity was significantly elevated in samples tested positive for DSA (94.2 cp/mL [84.7-264.1]) vs those tested negative (48.1 cp/mL [21.0-63.0]; p=0.024).

Dd-cfDNA performance compared to other biomarkers. Next, we sought to compare the performance of dd-cfDNA to conventional clinical tests used in assessing graft surveillance. We measured amylase and lipase levels in blood samples drawn concurrently with pancreatic biopsies. Although amylase levels did not significantly change between rejection and non-rejection groups (p=0.40), lipase was significantly higher in the rejection group compared to non-rejection (p=0.038). We compared the diagnostic ability of amylase, lipase, and dd-cfDNA (fraction and quantity) in distinguishing graft rejection from non-rejection based on histopathology results of pancreas grafts biopsies >45 days post-transplant. The calculated AUC of these biomarkers in discriminating pancreatic graft rejection from non-rejection were as follows: (dd-cfDNA quantity: 0.89); (dd-cfDNA fraction: 0.84); (lipase: 0.74); (amylase: 0.46). These data suggest that dd-cfDNA is superior to the marker assays traditionally used to discriminate pancreas rejection from non-rejection in SPKTx recipients. It is noteworthy to mention that attempts to combine dd-cfDNA and lipase simultaneously did not enhance the performance of dd-cfDNA.

Discussion: This pilot study is exploring for the first time the performance of dd-cfDNA to diagnose pancreas graft rejection in SPKTx recipients. We found that among stable patients, dd-cfDNA levels were elevated during the first 45 days after transplantation, compared to those performed after day 45. During this early period (<45 days), dd-cfDNA could not discriminate between P-BPAR and non-rejection. Of relevance, in biopsies performed >45 days post-transplant, dd-cfDNA quantity could discriminate between those with P-BPAR and those without acute rejection, with a sensitivity and specificity of 85% and 93%, respectively. Moreover, dd-cfDNA demonstrated better performance than the current available biomarkers, amylase and lipase. Of note, combining lipase and dd-cfDNA did not increase diagnostic accuracy compared to dd-cfDNA alone.

1. A method of amplifying and sequencing DNA, comprising:
(a) extracting cell-free DNA from a blood, plasma, serum or urine sample of a transplant recipient who has received transplantation of one or more organs, wherein the extracted cell-free DNA comprises donor-derived cell-free DNA and recipient-derived cell-free DNA;
(b) performing targeted amplification at 200-50,000 target loci in a single reaction volume using 200-50,000 primer pairs, wherein the target loci comprise polymorphic loci and non-polymorphic loci;
(c) sequencing the amplification products by high-throughput sequencing to obtain a sequencing reads and quantifying the amount of donor-derived cell-free DNA and the amount of total cell-free DNA based on the sequencing reads; and
(d) determining whether the amount of donor-derived cell-free DNA or a function thereof exceeds a cutoff threshold indicating transplant rejection or graft injury.

2. The method of claim 1, wherein the transplant recipient is a human subject.

3. The method of claim 1 or 2, wherein the transplant recipient has received a plurality of transplanted organs selected from pancreas, kidney, liver, heart, intestinal, thymus, and uterus.

4. The method of any of claims 1-3, wherein the one or more transplanted organs are from the same transplant donors.

5. The method of any of claims 1-3, wherein the one or more transplanted organs are from different transplant donors.

6. The method of any of claims 1-5, wherein the transplant recipient has received simultaneous transplantation of more than one organ.

7. The method of any of claims 1-5, wherein the transplant recipient has received sequential transplantation of more than one organs.

8. The method of claim 6, wherein the transplant recipient has received simultaneous transplantation of kidney and pancreas (SPK).

9. The method of claim 6, wherein the transplant recipient has received simultaneous transplantation of kidney and liver, simultaneous transplantation of kidney and heart, simultaneous transplantation of kidney and lung, simultaneous transplantation of pancreas and liver, or simultaneous transplantation of heart and lung.

10. The method of any of claims 1-9, wherein the cutoff threshold is a percentage of donor-derived cell-free DNA out of total cell-free DNA.

11. The method of any of claims 1-10, wherein the cutoff threshold is a copy number of donor-derived cell-free DNA or a function thereof.

12. The method of any of claims 1-11, wherein the cutoff threshold is a set of amount of donor-derived cell-free DNA.

13. The method of any of claims 1-12, wherein the cutoff threshold is a set concentration of donor-derived cell-free DNA.

14. The method of any of claims 1-13, wherein the targeted amplification comprises PCR, and the 200-50,000 primer pairs comprise forward and reverse PCR primers.

15. The method of any of claims 1-14, wherein the targeted amplification comprises performing targeted amplification at 1,000-10,000 target loci in a single reaction volume using 1,000-10,000 primer pairs to obtain amplification products.

16. The method of any of claims 1-15, wherein the target loci comprise single nucleotide polymorphisms (SNPs).

17. The method of any of claims 1-16, further comprising attaching tags to the amplification products prior to performing high-throughput sequencing, wherein the tags comprise sequencing-compatible adaptors.

18. The method of any of claims 1-17, further comprising attaching tags to the extracted cell-free DNA prior to performing targeted amplification, wherein the tags comprise adaptors for amplification.

19. The method of claim 17 or 18, wherein the tags comprise sample-specific barcodes, and wherein the method further comprises pooling the amplification products from a plurality of samples prior to high-throughput sequencing and sequencing the pool of amplification products together in a single run during the high-throughput sequencing.

20. The method of any of claims 1-19, further comprising repeating steps (a)-(d) longitudinally for the same transplant recipient, and determining a longitudinal change in the amount of donor-derived cell-free DNA or a function thereof in the transplant recipient.

21. The method of claim 20, further comprising adjusting immunosuppressive therapy based on the longitudinal change in the amount of donor-derived cell-free DNA or a function thereof in the transplant recipient.

22. The method of any of claims 1-21, wherein the method is performed without prior knowledge of donor genotypes.
